(19) United States
(12) Patent Application Publication
(10) Pub. No.: US 2014/0128648 A1
(43) Pub. Date: May 8, 2014

(54) HYDROCARBON CONVERSION PROCESS IN THE PRESENCE OF AN ACIDIC IONIC LIQUID WITH UPSTREAM HYDROGENATION

(71) Applicant: BASF SE, Ludwigshafen (DE)

(72) Inventors: Roman Prochazka, Mannheim (DE); Martin Bock, Ludwigshafen (DE); Steffen Tschirschwitz, Mannheim (DE); Gauthier Luc Maurice Averlant, Frankfurt (DE); Joni Joni, Sulzbach (DE); Markus Schmitt, Heidelberg (DE); Katharina Spuhl, Forest (BE); Jochen Bürkle, Mannheim (DE); Alois Kindler, Grunstadt (DE); Daniela Malkowsky, Speyer (DE); Stefan Bitterlich, Dirmstein (DE); Michael Hübner, Lampertheim (DE)

(73) Assignee: BASF SE, Ludwigshafen (DE)

(21) Appl. No.: 14/053,672

(22) Filed: Oct. 15, 2013

Related U.S. Application Data
(60) Provisional application No. 61/715,306, filed on Oct. 18, 2012.

(51) Int. Cl. C07C 5/13

(52) U.S. Cl. CPC C07C 5/13
USPC 585/253; 585/251

(57) ABSTRACT

The present invention relates to a process for hydrocarbon conversion in the presence of an acidic ionic liquid. The hydrocarbon conversion is preferably an isomerization, especially an isomerization of methylcyclopentane (MOP) to cyclohexane. Prior to the hydrocarbon conversion, a hydrogenation is performed, preference being given to hydrogenating benzene to cyclohexane. The cyclohexane obtained in the hydrogenation and/or isomerization is preferably isolated from the process. In a preferred embodiment of the present invention, the hydrogenation is followed and the hydrocarbon conversion, especially the isomerization, is preceded by distillative removal of low boilers, especially C₅-C₆-alkanes such as cyclopentane or isohexanes, from the hydrocarbon mixture used for hydrocarbon conversion.

This patent application claims the benefit of pending U.S. provisional patent application Ser. No. 61/715,306 filed on Oct. 18, 2012, incorporated in its entirety herein by reference.

The present invention relates to a process for hydrocarbon conversion in the presence of an acidic ionic liquid. The hydrocarbon conversion is preferably an isomerization, especially an isomerization of methylcyclopentane (MCP) to cyclohexane. Prior to the hydrocarbon conversion, a hydrogenation is performed, preference being given to hydrogenating benzene to cyclohexane. The cyclohexane obtained in the hydrogenation and/or isomerization is preferably isolated from the process. In a preferred embodiment of the present invention, the hydrogenation is followed and the hydrocarbon conversion, especially the isomerization, is preceded by distillative removal of low boilers, especially C₅-C₆-alkanes such as cyclopentane or isohexanes, from the hydrocarbon mixture used for hydrocarbon conversion.

Ionic liquids can be used in various hydrocarbon conversion processes; they are especially suitable as catalysts for the isomerization of hydrocarbons. A corresponding use of an ionic liquid is described, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

US-A 2003/0109767 discloses a process for isomerizing C₅-C₈ paraffin hydrocarbons (paraffins) in the presence of an ionic liquid as a catalyst. The ionic liquid comprises, as cations, nitrogen-containing heterocycles or nitrogen-containing aliphatics; the corresponding anions are derived from metal halides. The paraffins to be isomerized are linear alkanes such as n-hexane or n-octane and monosubstituted alkanes such as 3-methylhexane or mixtures thereof. The process described in US-A 2003/0109767 is intended to prepare paraffins having a relatively high degree of branching. In contrast, cyclohexane, for example, has a lower degree of branching compared to MCP. Moreover, US-A 2003/0109767 does not make any statements to the effect that any aromatics present in the starting mixture are hydrogenated prior to the isomerization.

In the isomerization process described in EP-A 1 403 236, the intention is likewise to obtain a relatively high degree of branching in the paraffins (hydrocarbons) to be isomerized in the presence of an ionic liquid. The isomerization process is additionally performed in the presence of cyclic hydrocarbons as additives and in a reaction medium, the cyclic hydrocarbons comprising a tertiary carbon atom as a structural unit, or being converted by the reaction medium to a corresponding compound having such a structural unit. Preference is given to using methylcyclohexane or dimethylcyclopentane as such cyclic hydrocarbon additives. The paraffins to be isomerized are linear alkanes such as n-butane or n-octane, and monomethyl-substituted alkanes such as 2-methylhexane. The ionic liquids are preferably based on nitrogen-containing heterocycles or nitrogen-containing aliphatics as cations, and on inorganic anions such as aluminum halides. EP-A 1 403 236 likewise does not contain any statements that any aromatics present in the starting mixture are hydrogenated prior to the isomerization.

US-A 2005/0082201 discloses a process for preparing gasoline with a low benzene content, wherein, in a first process step, a hydrocarbon mixture comprising benzene, olefins and sulfur compounds such as thiophenes is first fed into a distillation column, from which the low-boiling compounds are removed via the top, a benzene-containing fraction via a side draw and the high boilers from the column bottom. In a second process stage, the fraction obtained from the side draw is hydrogenated in the presence of a hydrogenation catalyst, converting benzene to cyclohexane and the thiophenes to hydrogen sulfide. The cyclohexane-containing mixture obtained in the second process stage is suitable for preparation of gasoline having a low benzene content. No isolation of the cyclohexane present therein or isomerization of MCP to cyclohexane is disclosed in US-A 2005/0082201.

WO 2010/027987 relates to a further process for reducing the concentration of benzene in a hydrocarbonaceous mixture. In a first separation stage, a benzene-containing fraction comprising benzene and other C₆ hydrocarbons is separated from a high boiler fraction comprising carbons having seven or more carbon atoms. The benzene-containing fraction is subsequently hydrogenated to obtain a hydrocarbon fraction having a reduced benzene content. The hydrogenation of benzene forms cyclohexane. WO 2010/027987 also does not contain any pointers that cyclohexane can be isolated from the mixture obtained in the hydrogenation; instead, this process product too is to be used for gasoline production. This document likewise does not disclose isomerization of MCP to cyclohexane.

U.S. Pat. No. 3,311,667 relates to a process for removing benzene from a mixture which is subsequently fed into an isomerization of MCP to cyclohexane. The hydrogenation involves hydrogenating benzene in the presence of a suitable catalyst, for example a metal catalyst on kieselguhr, with hydrogen to cyclohexane. The isomerization of MCP to cyclohexane is performed in the presence of metal halides such as acid-enhanced aluminum halide. U.S. Pat. No. 3,311,667, however, does not state that isomerization can also be accomplished using an acidic ionic liquid. Consequently, this document also does not make any statements that a hydrocarbon conversion, especially an isomerization, with acidic ionic liquids in the presence of aromatics, especially of benzene, is problematic.

EP-A 1 995 297 discloses a process and a corresponding apparatus for hydrogenation and decyclization of benzene and the isomerization of C₅-C₈ paraffins present in a mixture comprising at most 1% by weight of benzene. For hydrogenation of benzene, metallic catalysts can be used, suitable metals being the elements of the platinum group, tin or cobalt and molybdenum. For isomerization of the mixture obtained in the hydrogenation, which may comprise a residual amount of benzene, zeolites in particular are used as the catalyst. In the process described in EP-A 1 995 297, the parameters in the isomerization are adjusted such that opening of the cyclohexane rings obtained in the benzene hydrogenation to isoalkanes is achieved. The primary purpose of this process is thus not the preparation of cyclohexane but the preparation of alkanes having a high degree of branching. In addition, EP-A 1 995 297 also does not contain any statements that an acidic ionic liquid can also be used for isomerization, or that the removal of aromatics, particularly of benzene, prior to an isomerization is advantageous. A similar process to EP-A 1 995 297 is described in EP-A 1 992 673.

It is an object of the present invention to provide a novel process for performing a hydrocarbon conversion in the presence of an acidic ionic liquid.

The object is achieved by a process for hydrocarbon conversion, comprising the following steps:

a) hydrogenating a hydrocarbon mixture (HM1) comprising at least one aromatic and at least one nonaromatic hydrocarbon to obtain a hydrocarbon mixture (HM2) having a reduced amount of at least one aromatic compared to (HM1),
b) hydrocarbon conversion of at least one nonaromatic hydrocarbon present in (HM2) in the presence of an acidic ionic liquid.

### A BRIEF DESCRIPTION OF THE FIGURES

FIG. 1 illustrates the process according to the invention in a preferred embodiment of steps a) and b).

FIG. 2 shows a preferred embodiment of the present invention including a low boiler removal.

FIG. 3 shows general experimental setup, where feed and organics refer to the respective hydrocarbon mixtures (HM1) and (HM2).

### DETAILED DESCRIPTION OF THE INVENTION

Through the process according to the invention, it is advantageously possible to perform a hydrocarbon conversion, especially an isomerization, in the presence of an acidic ionic liquid, because the aromatics regularly present in hydrocarbon mixtures, especially benzene, can be completely or at least substantially removed by an upstream hydrogenation. Accordingly, the deactivation which otherwise occurs in the acidic ionic liquid used for hydrocarbon conversion, preferably for isomerization, especially for isomerization of MCP to cyclohexane, by aromatics, especially by benzene or other unsaturated compounds, is reduced or entirely avoided.

The removal of aromatics, especially of benzene, has the additional advantage that any distillative workup steps executed subsequently are facilitated because the formation of azeotropes of aromatics which otherwise occurs, for example benzene with saturated C₆-C₇-alkanes, is avoided.

This applies particularly to the embodiment described below and in FIG. 2, in which, in a first step, hydrocarbon mixture (HM1) comprising MCP and benzene is hydrogenated and then, for the purpose of removing a low boiler stream (LB), conducted into a distillation column (D1), the stream (HM2-(LB)) remaining after removal of the low boiler stream (LB) being passed into the isomerization, where the MCP is at least partly isomerized to cyclohexane. Since the object of (D1) is especially to remove lower boiling components than MCP, for example isohexanes, from MCP, this separation would be complicated by the presence of components which form azeotropes with MCP which are lower-boiling than MCP, this being the case for benzene. Thus, the hydrogenation of the benzene prior to the low boiler removal by means of the distillation column (D1) facilitates the separation which is the object of (D1).

If the target product of the process is cyclohexane and the compound to be hydrogenated is benzene, a further advantage of the process according to the invention is that the amount of the product obtained is increased by the cyclohexane obtained in the hydrogenation of benzene.

The process according to the invention for hydrocarbon conversion in the presence of an acidic ionic liquid with upstream hydrogenation is defined in detail hereinafter.

In the context of the present invention, in step a), a hydrocarbon mixture (HM1) comprising at least one aromatic and at least one nonaromatic hydrocarbon is hydrogenated to obtain a hydrocarbon mixture (HM2) having a reduced amount of at least one aromatic compared to (HM1). In other words, this means that, in step a), the aromatics present in the hydrocarbon mixture (HM1) are hydrogenated to obtain the corresponding nonaromatic hydrocarbons, preferably the fully saturated hydrocarbons which arise with retention of all carbon-carbon bonds. If other unsaturated compounds are present in the hydrocarbon mixture (HM1), for example olefins such as cyclohexene, these are likewise hydrogenated in step a) of the present invention. The hydrocarbon mixture (HM1) preferably comprises benzene as the aromatic and/or the hydrocarbon mixture (HM2) comprises an increased amount of cyclohexane compared to (HM1).

In principle, in the context of the present invention, it is possible to use any desired hydrocarbons as hydrocarbon mixture (HM1), provided that i) at least one of the hydrocarbons used is an aromatic which is hydrogenated in step a) and ii) at least one of the hydrocarbons used is a nonaromatic hydrocarbon which can be subjected in step b) (described below) to a hydrocarbon conversion, especially an isomerization, in the presence of an acidic ionic liquid. On the basis of his or her specialist knowledge, the person skilled in the art knows which hydrocarbons can be hydrogenated and which hydrocarbons can be subjected to a hydrocarbon conversion by means of acidic ionic liquids, and more particularly which hydrocarbons are isomerizable.

For example, hydrocarbon mixture (HM1) composed of two, three or even more hydrocarbons may be used, but it is also possible to use merely a mixture of a single aromatic, for example benzene, and of a single nonaromatic hydrocarbon, for example MCP. Preference is given in the context of the present invention to using hydrocarbon mixtures (HM1) which, apart from the two aforementioned components (hydrogenatable aromatic and convertible, preferably isomerizable, nonaromatic hydrocarbon), comprises further components, for example hydrocarbons, which are neither hydrogenatable nor can be subjected to a hydrocarbon conversion, more particularly an isomerization. Optionally, such mixtures may also comprise compounds which are not themselves hydrocarbons but are miscible therewith.

The individual components of the hydrocarbon mixture (HM1) may be present in any desired concentrations/ratios relative to one another. The hydrocarbon mixture (HM1) preferably comprises at least 90% by weight, preferably at least 95% by weight, of hydrocarbons having 5 to 8 carbon atoms, provided that i) at least one of the hydrocarbons used is a hydrogenatable aromatic and ii) at least one of the hydrocarbons used is a convertible nonaromatic hydrocarbon. The hydrocarbons may otherwise be saturated or unsaturated and/or cyclic, linear or branched. More particularly, the hydrocarbon mixture (HM1) comprises between 10% by weight and 60% by weight, more preferably between 20% by weight and 50% by weight, of MCP and/or between 1% by weight and 30% by weight, more preferably between 4% by weight and 20% by weight, of benzene.

In a preferred embodiment of the present invention, the hydrocarbon mixture (HM1) comprises benzene, methylcyclopentane (MCP) and at least one further compound selected from cyclohexane, cyclopentane, olefins and acyclic C₅-C₈-alkanes. In this embodiment, the further compounds preferably also comprise at least one low boiler selected from linear or branched C₅-alkanes, cyclopentane and linear or branched C₆-alkanes. The term “olefin” comprises, as well as linear, monounsaturated olefins such as pentene or hexene, also cyclic olefins, especially cyclohexene, and also dienes and cyclic dienes. In addition, the group of the C₅-C₈-alkanes also includes compounds having a standard boiling point >78° C., also called “high boilers” hereinafter.

More preferably, the hydrocarbon mixture (HM1) comprises benzene, methylcyclopentane (MCP) and at least one further hydrocarbon selected from cyclohexane, n-hexane, isohexanes, n-heptane, isoheptanes, methylcyclohexane or dimethylcyclopentanes.

If the hydrocarbon mixture (HM1) also comprises high boilers having a standard boiling point >78° C., especially dimethylpentanes (DMP), these high boilers, especially DMP, are preferably removed from the hydrocarbon mixture (HM1) prior to performance of step a) of the invention. The high boiler removal is thus preferably connected upstream of the hydrogenation. The high boiler removal is generally performed in a distillation apparatus, which is preferably a rectification column, preferably from the bottom of the corresponding distillation apparatus. The removal of the high boilers having a standard boiling point >78° C. from the hydrocarbon mixture (HM1) is preferably effected completely or virtually completely (down to 2% based on the amount of all high boilers, especially of all DMP isomers, present in the starting mixture).

The aforementioned embodiment of the present invention, which is also referred to as prior high boiler removal, is associated with an important advantage, especially when the high boilers having a standard boiling point >78° C. comprise DMP and when cyclohexane is prepared in the hydrogenation and/or preferably in the hydrocarbon conversion in the form of an isomerization. The reason for this is that, prior to the actual cyclohexane preparation process, the exceptionally complex separation, especially distillation, of DMP from the cyclohexane process product can be avoided. This is especially true when the DMP is 2,4-dimethylpentane (2,4-DMP) and the latter is present in the starting mixture in a concentration >100 ppm. This distinctly reduces the energy intensity and apparatus complexity in the preparation of pure or high-purity cyclohexane.

In the context of the present invention, the hydrogenation of the hydrocarbon mixture (HM1) is effected in an apparatus (V) suitable for this purpose, this apparatus preferably comprising at least one hydrogenation reactor (HR). In the apparatus (V), benzene is preferably hydrogenated to cyclohexane, the hydrogenation preferably being effected using hydrogen. It is additionally preferable that the hydrogenation is effected in the liquid phase.

The hydrogenation of at least one aromatic in step a), preferably of benzene to cyclohexane, is generally performed in the presence of a suitable catalyst. Suitable catalysts are in principle all catalysts known to those skilled in the art for this purpose, for example a metal catalyst on kieselguhr according to U.S. Pat. No. 3,311,667 or metallic catalysts according to EP A 1 995 297, wherein the metals used with preference are the elements of the platinum group, tin or cobalt and molybdenum.

Preference is given to performing the hydrogenation in the presence of a catalyst comprising, as an active metal (also referred to as metal component or active component), at least one element of groups 8 to 10 of the Periodic Table of the Elements (PTE), for example iron, cobalt, nickel or ruthenium (corresponds to transition group VIIIB of the CAS version of the PTE), especially nickel or ruthenium. It is additionally preferable that the active metal is applied to a support material (support). Suitable supports are in principle all supports known to those skilled in the art, for example SiO₂-containing, zirconia-containing or alumina-containing supports. Particular preference is given to using a catalyst comprising nickel as an active metal on an alumina-containing support.

The hydrogenation as such is executed and operated in a manner known per se to those skilled in the art, preference being given to a combination of a main reactor operated in an optionally cooled circuit (recycling of a portion of the mixture flowing out of the reactor into the mixture flowing into the reactor, with optional positioning of the cooling unit upstream or downstream of said feed) and a downstream postreactor operated in straight pass, i.e. without recycling. In this case, the apparatus (V) thus comprises two hydrogenation reactors (HR).

The hydrogenation reactors (HR) are preferably designed as fixed bed reactors without internal cooling. In this case, the hydrogenation is preferably operated such that the temperature differential between entering and exiting mixture is monitored continuously and, when this value falls below a particular target value, the entrance temperature is raised. It is additionally preferable that the hydrogenation reactors are operated in trickle mode.

It is additionally preferable that the hydrogenation is followed downstream by an apparatus in which decompression is effected to a pressure below the pressure established in the postreactor. This affords a gas stream which comprises hydrogen dissolved beforehand in the hydrocarbon mixture and is in any case compressed and recycled into at least one of the hydrogenation reactors (HR).

The hydrogenation is preferably performed at a temperature between 50 and 200° C., more preferably between 100 and 180° C., and/or a pressure between 10 and 300 bar abs., more preferably between 30 and 200 bar abs.

It is additionally preferable in the process according to the invention that the overall conversion of the aromatics, especially of the benzene (and of any other unsaturated compounds present in the hydrocarbon mixture (HM1)), in the hydrogenation is at least 90%, more preferably 99%, and/or the residual content of the aromatics, especially of the benzene (and of any other unsaturated compounds present in the hydrocarbon mixture (HM1)), in the hydrocarbon mixture (HM2) is 1% by weight, preferably at most 0.1% by weight, more preferably at most 0.01% by weight.

Owing to the hydrogenation, in step a) of the invention, the hydrocarbon mixture (HM2) is obtained, the composition of which differs from the hydrocarbon mixture (HM1) primarily with respect to the hydrogenated compounds. The hydrocarbon mixture (HM2) thus comprises at least one hydrocarbon formed by hydrogenation of an aromatic and at least one nonaromatic hydrocarbon which had already been present in (HM1). In addition, the hydrocarbon mixture (HM2) comprises all other components as per hydrocarbon mixture (HM1) which are not chemically altered in the hydrogenation, and any hydrocarbons formed by hydrogenation of olefins or dienes. If the aromatic present in the hydrocarbon mixture (HM1) is benzene, the hydrocarbon mixture (HM2) correspondingly comprises cyclohexane.

The hydrocarbon mixture (HM2) preferably comprises cyclohexane, MCP, not more than 0.1% by weight of aromatics and possibly at least one further compound selected from olefins and acyclic C5-C8-alkanes. More preferably, the hydrocarbon mixture (HM2) comprises cyclohexane, methylcyclopentane (MCP) and at least one further hydrocarbon selected from cyclohexane, n-hexane, isohexanes, n-heptane, isoheptanes, methylcyclohexane or dimethylcyclopentanes.

In step b) of the process according to the invention, a hydrocarbon conversion of at least one nonaromatic hydrocarbon present in (HM2) is effected in the presence of an acidic ionic liquid.

Hydrocarbon conversions as such are known to those skilled in the art. The hydrocarbon conversion is preferably selected from an alkylation, a polymerization, a dimerization, an oligomerization, an acylation, a metathesis, a polymerization or copolymerization, an isomerization, a carbonylation or combinations thereof. Alkylations, isomerizations, polymerizations etc. are known to those skilled in the art. Especially preferably in the context of the present invention, the hydrocarbon conversion is an isomerization.

In the context of the present invention, the hydrocarbon conversion is preferably effected in the presence of an acidic ionic liquid having the composition K1AlₙX₍₃ₙ₊₁₎ where K1 is a monovalent cation, X is halogen and 1≦n≦2.5. Such acidic ionic liquids are known to those skilled in the art; they are disclosed (alongside further ionic liquids), for example, in WO 2011/069929. For example, mixtures of two or more acidic ionic liquids may be used, preference being given to using one acidic ionic liquid.

K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The acidic ionic liquid more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having 1 to 10 carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of 1 to 3 carbon atoms. The heterocyclic cation is preferably an imidazolium ion or a pyridinium ion.

The acidic ionic liquid especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred acidic ionic liquids are trimethylammonium chloroaluminate and triethylammonium chloroaluminate.

The acidic ionic liquid used in the context of the present invention is preferably used as a catalyst in the hydrocarbon conversion, especially as an isomerization catalyst.

Furthermore, in the hydrocarbon conversion, especially in the isomerization, in addition to the acidic ionic liquid, it is also possible to use a hydrogen halide (HX) as a cocatalyst. The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described acidic ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (HCl).

The hydrocarbon conversion can in principle be performed in all apparatuses known for such a purpose to the person skilled in the art. The corresponding apparatus is preferably a stirred tank or a stirred tank cascade. A “stirred tank cascade” means that two or more, for example three or four, stirred tanks are connected in succession (in series).

It is additionally preferable in the context of the present invention that the hydrocarbon conversion is performed, preferably as an isomerization, in a dispersion (D1), with dispersion of phase (B) in phase (A) in dispersion (D1), the volume ratio of phase (A) to phase (B) being in the range from 2.5 to 4:1 [vol/vol], phase (A) comprising >50% by weight of at least one acidic ionic liquid, and phase (B) comprising >50% by weight of at least one nonaromatic hydrocarbon. It is additionally preferable that the dispersion (D1) additionally comprises HCl and/or gaseous HCl is introduced into the dispersion (D1).

This embodiment of the present invention, in which the hydrocarbon conversion, especially the isomerization, is performed with a specific volume ratio of phase (A) to phase (B) in the range from 2.5 to 4:1 [vol/vol], can achieve a higher space-time yield, which constitutes a further advantage of the process according to the invention. Due to this optimization, the apparatus complexity for performance of the process can also be reduced; for example, the apparatus in which the hydrocarbon conversion, especially the isomerization, is performed can be kept small. It is thus possible to use smaller or fewer reactors.

As already explained above, due to the hydrocarbon conversion in the presence of an acidic ionic liquid and optionally of a hydrogen halide (HX), the chemical structure of at least one of the nonaromatic hydrocarbons used is altered. The hydrocarbons obtained in the hydrocarbon conversion are present in a hydrocarbon mixture (HM2b). Mixture (HM2b) thus differs in terms of (chemical) composition and/or amount of the hydrocarbons present therein from the corresponding hydrocarbon mixture (HM2) present prior to the hydrocarbon conversion, especially prior to the isomerization. The hydrocarbon mixture (HM2) has already been defined above in connection with step a).

Since the hydrocarbon conversion to be performed in such hydrocarbon conversions, especially in isomerization processes, frequently does not proceed to an extent of 100% (i.e. to completion), the product generally still also comprises the hydrocarbon with which the hydrocarbon conversion has been performed (in a smaller amount than before the isomerization). If, for example, MCP is to be isomerized to cyclohexane, the isomerization product frequently comprises a mixture of cyclohexane and (in a smaller amount than before the isomerization) MCP.

In the context of the present invention, the hydrocarbon conversion is preferably an isomerization in which methylcyclopentane (MCP) is isomerized to cyclohexane.

If the hydrocarbon conversion in the context of the present invention is an isomerization, the isomerization is preferably performed as follows. The performance of an isomerization of hydrocarbons in the presence of an ionic liquid as a catalyst and optionally a hydrogen halide as a cocatalyst is known to those skilled in the art. The hydrocarbons and the ionic liquid in the isomerization preferably each form a separate phase, though portions of the ionic liquid may be present in the hydrocarbon phase and portions of the hydrocarbons in the ionic liquid phase. The hydrogen halide, especially hydrogen chloride, is introduced (if present), preferably in gaseous form, into the apparatus for performance of the isomerization. The hydrogen halide may, at least in portions, be present in the two aforementioned liquid phases and in a gaseous phase which is preferably additionally present.

The isomerization is preferably performed at a temperature between 0° C. and 100° C., especially preferably at a temperature between 30° C. and 60° C. It is additionally preferred that the pressure in the isomerization is between 1 and 20 bar abs. (absolute), preferably between 2 and 10 bar abs.

The isomerization is preferably performed in the apparatus in such a way that two liquid phases and one gas phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components from which the ionic liquid is formed in solid form, for example AlC13. The pressure and composition of the gas phase are set here such that the partial pressure of the gaseous hydrogen halide, especially of HCl gas, in the gas phase is between 0.5 and 20 bar abs. (absolute), preferably between 1 and 10 bar abs.

It is additionally preferable in the process according to the invention that cyclohexane is isolated from the mixture obtained in the hydrocarbon conversion, especially when the hydrocarbon conversion is an isomerization. In general, in the process according to the invention, after the hydrocarbon conversion, cyclohexane is isolated in a purity of at least 98% by weight, preferably at least 99.5% by weight, more preferably 99.9% by weight. The cyclohexane can be isolated by methods known to those skilled in the art, for example using one or more distillation columns into which the output from the apparatus in which the hydrocarbon conversion, especially the isomerization, has been performed is introduced. Optionally, cyclohexane which has been obtained in step a) of the invention may already be isolated from the hydrocarbon mixture (HM2) after the hydrogenation and prior to the hydrocarbon conversion.

Preferably, in the context of the present invention, prior to any distillative removal/isolation of the cyclohexane after the hydrocarbon conversion, especially after the isomerization, additional purification steps are performed with the output from the hydrocarbon conversion, preferably the isomerization. These purification steps may, for example, be a neutral and/or alkaline wash, which can be performed in one or more stages. Additionally or alternatively to the wash, it is also possible to use specific apparatuses, for example distillation or rectification apparatuses, in order, for example, to separate hydrogen halide present from the hydrocarbons. Such apparatuses also comprise apparatuses for one-stage evaporation, especially for flash evaporation. Additionally or alternatively, it is also possible to connect phase separation units, preferably phase separators, upstream of the aforementioned specific apparatuses, especially in order to separate the acidic ionic liquid from the hydrocarbons.

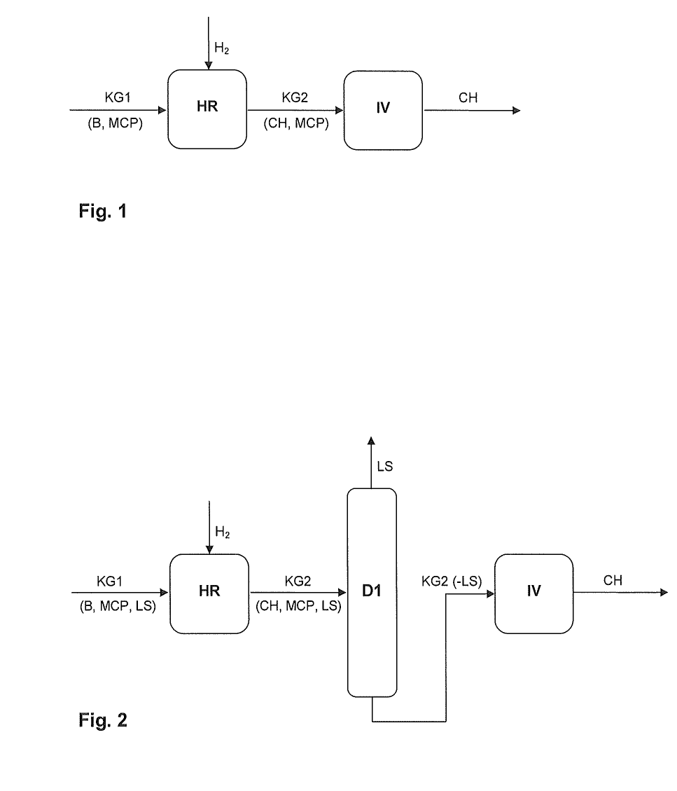

FIG. 1 once again illustrates the process according to the invention in a preferred embodiment of steps a) and b). CH means cyclohexane, B means benzene, and the bracketed expressions indicate the components most relevant to the process and/or the main components of the respective stream. In the embodiment according to FIG. 1, the hydrocarbon mixture (HM1) is first hydrogenated in at least one hydrogenation reactor (HR) using hydrogen. The aromatic present in the hydrocarbon mixture (HM1) is benzene and the nonaromatic hydrocarbon present is MCP; it is also possible (as described above in connection with (HM1)) for further hydrocarbons to be present in the hydrocarbon mixture (HM1). In step a), the benzene is converted completely or virtually completely to cyclohexane (to obtain the hydrocarbon mixture (HM2)).

The hydrocarbon conversion in the configuration according to FIG. 1 is an isomerization. The isomerization of the hydrocarbon mixture (HM2), in which MCP is isomerized in the presence of an acidic ionic liquid to cyclohexane, is effected in an isomerization apparatus (IV) suitable for this purpose. The isomerization is preferably performed in a stirred tank or a stirred tank cascade. Cyclohexane is subsequently isolated from the isomerization product, for example using one or more distillation columns into which the output from the isomerization apparatus (IV) is introduced.

In a preferred embodiment of the present invention, the hydrogenation in step a) is followed and the hydrocarbon conversion, especially the isomerization, in step b) is preceded by removal of low boilers, especially C₅-C₆-alkanes such as cyclopentane or isohexanes, from the hydrocarbon mixture (HM2) used for hydrocarbon conversion. The removal is preferably effected by means of distillation. This (preferably distillative) removal is also referred to hereinafter as “low boiler removal”, which can be performed in apparatuses known to those skilled in the art, especially using a distillation column (D1).

The low boiler removal involves, in accordance with the invention, preferably distillative separation of low boilers from the rest of the hydrocarbons in the hydrocarbon mixture (HM2) which has a reduced amount of at least one aromatic compared to the hydrocarbon mixture (HM1) and additionally comprises at least one nonaromatic hydrocarbon. The hydrocarbon mixture (HM2) depleted of the low boilers is subsequently sent to the hydrocarbon conversion, especially the isomerization, in step b) of the present invention. The hydrocarbon mixture (HM2) depleted of the low boilers is removed, preferably from the bottom of the corresponding distillation column.

The low boiler removal is preferably performed in such a way that the hydrocarbon conversion in step b) is preceded by distillative removal of at least one compound selected from linear or branched C₅-alkanes, cyclopentane and linear or branched C₆-alkanes from hydrocarbon mixture (HM2). More preferably, isohexanes are separated by distillation from the hydrocarbon (HM2). The low boilers are preferably removed from the top of the corresponding distillation column.

The above-described preferred embodiment of the present invention including a low boiler removal is additionally illustrated below in a preferred embodiment in conjunction with FIG. 2. In FIG. 2, the abbreviations, arrows and other symbols have similar meanings to those explained above for FIG. 1. In the embodiment according to FIG. 2, the benzene-containing hydrocarbon mixture (HM1) is first hydrogenated, with complete or virtually complete conversion of the benzene to cyclohexane (to obtain the hydrocarbon mixture (HM2)). The hydrocarbon conversion in the configuration according to FIG. 2 is an isomerization. LB means low boilers; the low boilers are preferably linear or branched C₅-alkanes, cyclopentane and/or linear or branched C₆-alkanes, especially isohexanes.

In the distillation column (D1), the low boilers are removed from the hydrocarbon mixture (HM2) as stream (LB), stream (LB) boiling at a lower temperature than (HM2). Stream (LB), compared to (HM2), is preferably enriched in isohexanes and/or cyclopentane and depleted of MCP. The hydrocarbon mixture (HM2) depleted of/reduced by stream (LB), which is referred to in FIG. 2 as “HM2-(LB)”, boils at a higher temperature than (HM2). Stream (HM2-(LB)) is preferably depleted of isohexanes and/or cyclopentane and enriched in MCP compared to (HM2).

The low boiler removal is preferably executed and operated in such a way that stream (LB) comprises less than 5% by weight, more preferably less than 2.5% by weight, of MCP and stream (HM2-(LB)) comprises less than 10% by weight, more preferably less than 5% by weight, of isohexanes.

Stream (LB) can, for example, be introduced into a steamcracker as what is called cracker cofeed, while stream (HM2-(LB)) is conducted into the hydrocarbon conversion, preferably into the isomerization stage. Optionally, within the low boiler removal, it is possible to draw off a further stream depleted of isohexanes and enriched in components having a lower boiling point than the isohexanes, for example chlorinated paraffins having <4 carbon atoms per molecule, compared to stream (LB).

The examples which follow illustrate the invention.

### EXAMPLES

The adverse influence of benzene on the isomerization of methylcyclopentane into cyclohexane is investigated in the following experiment. This shows up the need for an upstream hydrogenation of benzene to eliminate this disruptive component.

The following composition is chosen as hydrocarbon mixture (HM2):
methylcyclopentane at 20% by weight
cyclohexane at 50% by weight
hexane at 28%°
isohexanes (technical-grade mixture) at 2% by weight

The following acidic ionic liquid (IL) is used for the isomerization:

(CH₃)₃NHAlₙCl₃ₙ₊₁ with n=1.82 as per elemental analysis.

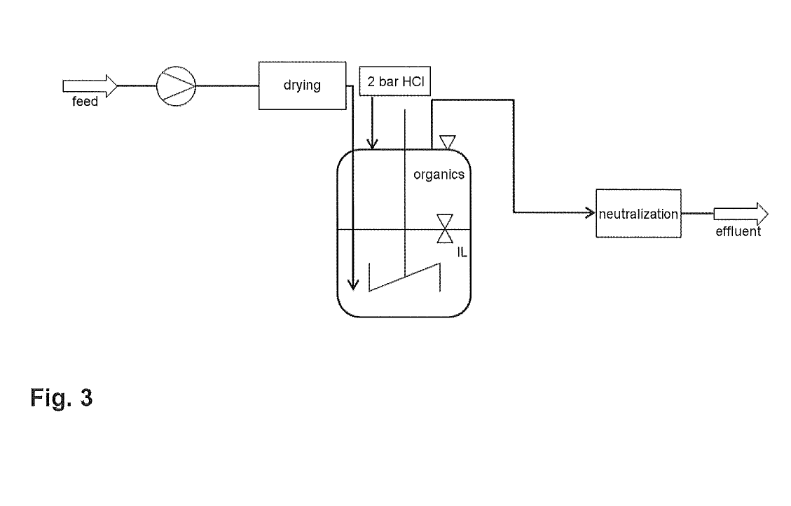

The general experimental setup is depicted in FIG. 3, where feed and organics refer to the respective hydrocarbon mixtures (HM1) and (HM2). The experimental setup is for a continuous process, i.e., it is a continuous plant.

### Example 1

A 260 ml pressure vessel made of glass with a heatable jacket is initially charged with 180 g of IL (130 ml). The reaction temperature is set to 50° C. at a stirrer speed of 1000 rpm and a continuous HCl supply of 1.038 standard L/h. The hydrocarbon mixture (HM2) is continuously added at a rate of 65 g/h. The corresponding amount of converted hydrocarbon mixture is concurrently removed from the upper part of the vessel and analyzed.

When the hydrocarbon mixture (HM2) is used, a constant conversion of methylcyclopentane can be observed over a period of more than 1000 h without visual change in the separating layer between the organics (hydrocarbon mixture (HM2) and the IL.

### Comparative Example 2

The hydrocarbon mixture (HM1) is used instead of the hydrocarbon mixture (HM2) as follows:

Following the 1000 h of operation with (HM2), 30 ppm of benzene are initially added to (HM2) (to obtain HM1)) and fed into the continuous plant for a prolonged period. No effects on the conversion of the reaction can be observed, and the measured absolute fraction of cyclohexane (CH) remains constant. Even increasing the benzene fraction to 50, 80, 150 or 200 ppm of benzene did not result in any observable changes in the conversion of the reaction.

However, over time, problems did increasingly occur with the plant in the sense that solid material was formed and exported, which leads to pressure increases and/or cloggages, which accordingly denotes a deactivation of the acidic IL used as catalyst. True, the absolute measured fraction of CH in the effluent remains high, but the reaction no longer performs consistently well and major fluctuations are observed. On addition of 400 ppm of benzene, a thick layer of crud then forms within two days between the IL phase and the organics phase and impeccable phase separation is no longer achievable. Nonetheless, MCP conversion is as high as ever.

On addition of 800 ppm of benzene, the problems with solid material being exported increase and the layer of crud spreads quickly throughout the entire reactor volume, so there is no longer any visible phase boundary. The experiment then had to be discontinued. However, neither the conversion of the reaction nor the elemental analysis of the IL after the end of the reaction showed any abnormalities. Analysis of the reaction effluents illustrated that only small amounts (1-5 ppm) of benzene remain in the system. Therefore, the effects observed can be reduced to interphase formation and the associated physico-chemical problems, i.e., deactivation of the acidic IL used as catalyst.

To verify that the formation of the layer of crud was not a consequence of running the system for a long time with different amounts of benzene, the reaction kettle was emptied, fresh IL was introduced and the plant was started up directly with a feed containing 400 ppm of benzene. Interphase formation did reoccur and within a few days the reactor was half filled with a thick emulsion.

1. A process for hydrocarbon conversion, comprising the following steps:
a) hydrogenating a hydrocarbon mixture (HM1) comprising at least one aromatic and at least one nonaromatic hydrocarbon to obtain a hydrocarbon mixture (HM2) having a reduced amount of at least one aromatic compared to (HM1),
b) hydrocarbon conversion of at least one nonaromatic hydrocarbon present in (HM2) in the presence of an acidic ionic liquid.

2. The process according to claim 1, wherein the hydrocarbon conversion is selected from an alkylation, a polymerization, a dimerization, an oligomerization, an acylation, a metathesis, a polymerization or copolymerization, an isomerization, a carbonylation or combinations thereof.

3. The process according to claim 2, wherein the hydrocarbon conversion is an isomerization, preferably an isomerization of methylcyclopentane (MCP) to cyclohexane.

4. The process according to any of claims 1 to 3, wherein the aromatic present in the hydrocarbon mixture (HM1) is benzene and/or the hydrocarbon mixture (HM2) comprises an increased amount of cyclohexane compared to (HM1).

5. The process according to any of claims 1 to 4, wherein the hydrogenation of the hydrocarbon mixture (HM1) is performed in the presence of a catalyst comprising, as an active metal, at least one element of groups 8 to 10 of the Periodic Table of the Elements, especially nickel or ruthenium.

6. The process according to any of claims 1 to 5, wherein a catalyst comprising nickel as the active metal on an alumina-containing support is used.

7. The process according to any of claims 1 to 6, wherein the hydrocarbon mixture (HM1) comprises benzene, methylcyclopentane (MCP) and at least one further compound selected from cyclohexane, cyclopentane, olefins and acyclic C₅-C₈-alkanes.

8. The process according to any of claims 1 to 7, wherein the hydrocarbon mixture (HM2) comprises cyclohexane, MCP, not more than 0.1% by weight of aromatics and possibly at least one further compound selected from olefins and acyclic C₅-C₈-alkanes.

9. The process according to any of claims 1 to 8, wherein the acidic ionic liquid comprises, as a cation, an at least partly alkylated ammonium ion or a heterocyclic cation and/or, as an anion, a chloroaluminate ion having the composition AlₙCl₍₃ₙ₊₁₎ where 1<n<2.5.

10. The process according to any of claims 1 to 9, wherein the hydrocarbon conversion is performed, preferably as an isomerization, in a dispersion (D1), with dispersion of phase (B) in phase (A) in dispersion (D1), the volume ratio of phase (A) to phase (B) being in the range from 2.5 to 4:1 [vol/vol], phase (A) comprising >50% by weight of at least one acidic ionic liquid, and phase (B) comprising >50% by weight of at least one nonaromatic hydrocarbon.

11. The process according to claim 10, wherein D1 additionally comprises HCl and/or gaseous HCl is introduced into dispersion (D1).

12. The process according to any of claims 1 to 11, wherein the hydrocarbon conversion is performed as an isomerization in a stirred tank or a stirred tank cascade.

13. The process according to claim 12, wherein the isomerization is conducted in a stirred tank or a stirred tank cascade at a temperature of 30 to 60° C. and/or a pressure of 2 to 10 bar abs.

14. The process according to any of claims 1 to 13, wherein the hydrocarbon conversion in step b) is preceded by distillative removal of at least one compound selected from linear or branched C₅-alkanes, cyclopentane and linear or branched C₆-alkanes from hydrocarbon mixture (HM2).

15. The process according to any of claims 1 to 14, wherein the hydrocarbon conversion is an isomerization and cyclohexane is isolated from the mixture obtained in the isomerization.
